(19) United States
(12) Patent Application Publication
(10) Pub. No.: US 2007/0072219 A1
(43) Pub. Date: Mar. 29, 2007

(54) Detection method of homologous sequences differing by one base on a microarray

(76) Inventors: Jose Remacle, Malonne (BE); Francoise De Longueville, Natoye (BE); Soumya Pastoret, Liege (BE); Vincent Bertholet, Jambes (BE)

Correspondence Address: KNOBBE MARTENS OLSON & BEAR LLP, 2040 MAIN STREET, FOURTEENTH FLOOR, IRVINE, CA, 92614, US

(21) Appl. No.: 11/520,858

(22) Filed: Sep. 12, 2006

(30) Foreign Application Priority Data
Sep. 13, 2005 (EP) 05447202.2

(51) Int. Cl. C12Q 1/68; G06F 19/00; C12P 19/34

(52) U.S. Cl. 435/6; 435/91.2; 702/20

(57) ABSTRACT

The present invention is related to a detection method and kit of homologous sequences differing by one base on a microarray.

### RELATED APPLICATIONS

This application claims the benefit of European application No. 05447202.2, filed Sep. 13, 2005.

### FIELD OF THE INVENTION

The present invention is related to a method and kit or system comprising reagents and means for the identification (detection) of possible mutations (SNPs) in gene(s) or organism genome based on amplification of homologous sequence followed by detection on array.

The invention is especially suited for the simultaneous identification and/or quantification of multiple mutations in the same gene nucleotide sequence or same organism genome.

The present invention is well adapted for diagnostic and analytical assay.

### BACKGROUND OF THE INVENTION

The early methods to type single nucleotide polymorphisms SNPs include the following techniques SSCP, RFLP, AS-PCR, sequencing making genotyping judgment coupled with gel electrophoresis. These methods are unfit for large scale screening due to the limitation of detection methods. However multiple analyses of mutations have been facilitated by using DNA microarray based method for questioning each particular position where mutations may occur in a particular sequence. Also the microarray based mutation detection can be extended to multiple different sequences, like typical exons of the same gene which can not usually be sequenced at once given the distance between the exons.

In the most common method, the gene or genome questioned for possible mutation is first amplified and then copied into ribonucleotide sequences in order to be cut into pieces which are then hybridized on oligonucleotides sequences present on the array. The presence of a mutation is considered according to the ratio of the signal of the oligonucleotide having the mutation compared to the wild type.

Several publications exist on this technology. An allele specific oligonucleotide (ASO) based microarray was made for the screening of 4 mutant alleles of CYP2C9 (Wen S. Y. et al. 2003, World J. Gastroenterol., 9:1342-1346). Pairs of probes with one base difference for SNP discrimination were immobilized on glass slides. Genotype was determined by calculation of the signal ratio of match to mismatch probes. The signal intensity ratio value above 4 or below 2.5 is considered a critical limit for genotyping judgment. When ratio values were between 2.5 and 4, samples were re-genotyped.

The U.S. Pat. No. 6,410,229 provides an array of nucleic acid probes for SNP detection in RNA transcripts. Quantification of the hybridization is obtained by comparing binding of matched and control probes. The patent application WO9729212 provides a method for identifying a genotype of an organism using an array comprising capture probes complementary to reference DNA or RNA sequences from another organism (for example, using oligonucleotide sequences based on the Mycobacterium tuberculosis rpoB gene). Genotyping is based on an overall hybridization pattern of the target to the array.

The U.S. Pat. No. 5,858,659 provides an array comprising detection blocks of probes, each block including four groups of capture probes to question. a single base (e.g. use of blocks of 40 oligonucleotides of 20 bases to question one polymorphic base). First and second groups of capture probes are complementary to the target nucleic acid sequence having first and second variants of the polymorphic bases. Third and fourth groups of probes, have a sequence identical to first and second groups of probes, except that they include mono-substitutions of positions in said sequence that are within n bases of the polymorphic base.

If the method is working, it has some drawbacks since different capture probes have different affinity for target sequences and the ratios between the mutated to non mutated capture probes varies a lot from one mutation to the other. Also the determination of common hybridization conditions is a problem with some capture probes having better discrimination than the others. The consequence is a very heterogeneous pattern of ratios and sometimes a difficulty to determine whether the organism is homozygote or heterozygote for the mutations at specific loci.

Two improvements have been recently proposed to this method. The US patent application 2005/0089877, provides a method for genotyping DNA sequence on chips with a wild-perfect match and a mutant perfect match probe. The method proposes a genotyping algorithm for the optimization of the capture probes and a statistical robust method. The algorithm is based on the analysis of data coming from a hybridization of an identified standard nucleic acid and from a genotyping of the unknown target by substituting input vectors into the genotyping algorithm.

The U.S. Pat. No. 6,852,488 is related to a sequencing method of a target sequence, but with particular detection of mutation in the target sequence. The method is based upon the use of a core known sequence with high affinity for the target sequence. The method proposes a selection of a probe by evaluating the binding characteristics of all the probes having a single mismatch as compared to a core probe. If the single base mismatch probes exhibit characteristic binding or affinity pattern, then the core probe is exactly complementary to at least a portion of the target sequence. This method allows mutation detection in a target sequence by a comparison of binding affinity of a known core probe for the target sequence with the binding affinity of the probe having a single nucleotide variation. This selection method of the probe is based on the experimental screening of capture probes with selection of one with high affinity binding.

However, these two methods require multiple experimental steps and are therefore complicated and time consuming. Furthermore, they are not fit for the development of large number of mutation detections on microarray since they require experimental optimization for each mutation and they use a different calculation process for obtaining a final result related to each mutation. They also do not provide an a priori solution for developing biochips for the detection of multiple SNPs in one organism genome.

### SUMMARY OF THE INVENTION

The present invention provides an original and easy solution to determine a presence or absence of at least 3 and preferably 5 and still preferably 20 single nucleotide polymorphisms or SNP (mutated base 1) at given loci of gene nucleotide sequence(s) (3) of an organism (including the human) which means the identification or detection of several homologous sequences (7,7′) present in the said sequence(s) (3) differing by one base (1) and comprising the steps of:
possibly extracting the gene nucleotide sequence(s) (3) from said organism (including a human);
amplifying the organism obtained from homologous gene nucleotide sequences or a portion thereof containing different loci into a first set(s) of target nucleotide sequences (homologous sequences 7 or 7′), and;
amplifying a second fragment of gene nucleotide sequence(s) being non mutated into a second set of target nucleotide sequences (8);
putting into contact the first set(s) of target nucleotide sequences (7 or 7′) upon an array of capture probes (9,9′, 10) (bound to a solid support (22) surface) wherein the capture probes (9,9′or 10) have similar physical or chemical properties and wherein the said array comprises a series of pair of capture probes (9 and 9′) having the same specific hybridization sequence complementary to a portion of the different target nucleotide sequences (7 and 7′) but differing by a single base (20), this single base (20) being complementary to the mutated base (1) present in the locus sequence to be characterized;
putting into contact the second set of target nucleotide sequences (8) with a complementary capture probe (10) having a specific hybridization sequence complementary to a portion of the said target nucleotide sequence (8) but differing by a single base (21);
detecting and/or quantifying a signal value of hybridization of the first set of target nucleotide sequences (7 or 7′) and a signal value of hybridization of the second set of target nucleotide sequences (8);
determining the presence of the nucleotide base (1) at the different said loci, when the signal values of detection of the first set of target sequence(s) (7 and/or 7′) upon their corresponding capture probes (9 or 9′) is higher or equal to a cut off signal value calculated from the signal value of detection of the second set of the target sequences (8) upon corresponding specific mutated capture probes (10); and
determining the presence of single nucleotide polymorphisms (of the mutated base 1) in the different (said) loci.

In the method according to the invention, this detection is particularly adapted for the identification of multiple single nucleotide polymorphisms or multiple mutations (multiple SNPs) present at different gene locus. The method also provides tools and means to determine whether an organism is heterozygote (different alleles) or homozygote (same alleles) at a particular gene locus.

Preferably, this detection or characterization is obtained upon the same array. Furthermore, the step of comparing the signal value of hybridization between the different sets of targets and their corresponding capture probes is preferably made upon the same array. The capture probes are preferably present at specific locations of a solid support surface (22) forming an array.

Definitions

Unless otherwise defined, all technical and scientific terms used herein have the same meaning as commonly understood by one person ordinary skilled in the art to which this invention belongs.

The terms “nucleotide sequence, array, target (and capture) nucleotide sequence, bind substantially, hybridizing specifically to, background, quantifying” are as described in WO97/27317, which is incorporated herein by way of reference.

The terms “nucleotide triphosphate, nucleotide, primer sequence” are those described in the European patent application EP1096024 incorporated herein by reference.

The term “gene” means fundamental physical and functional unit of heredity, which carries information from one generation to the next; a segment of DNA located in a specific site on a chromosome that encode a specific functional product. The DNA segment is composed of transcribed region and a regulatory sequence that makes transcription possible (regions preceding and following the coding DNA as well as introns between the exons).

The term “locus” means the position of the single nucleotide polymorphism (SNP) upon the sequence of the gene.

“Homologous sequences” mean nucleotide sequences having a percentage of nucleotides identical at corresponding positions which is higher than in purely random alignments. Two sequences are considered as homologous when they show between them a minimum of homology (or sequence identity) defined as the percentage of identical nucleotides found at each position compared to the total nucleotides, after the sequences have been optimally aligned taking into account additions or deletions (like gaps) in one of the two sequences to be compared. The degree of homology (or sequence identity) can vary a lot as homologous sequences may be homologous only in one part, a few parts or portions or all along their sequences. Nucleotide sequences differing by only one base are sequences highly homologous and qualified as single nucleotide polymorphisms (SNPs). The parts or portions of the sequences that are identical in both sequences are said conserved. Protein domains which present a conserved three dimensional structure are usually coded by homologous sequences and even often by a unique exon. The sequences showing a high degree of invariance in their sequences are said to be highly conserved and they present a high degree of homology.

Methods of alignment of sequences are based on local homology algorithms which have been computerized and are available as for example (but not limited to) Clustal®, (Intelligenetics, Mountain Views, Calif.), or GAP®, BESTFIT®, FASTA® and TFASTA® (Wisconsin Genetics Software Package, Genetics Computer Group Madison, Wis., USA) or Boxshade®.

The term “consensus sequence” is a sequence determined after alignment of the several homologous sequences to be considered (calculated as the base which is the most commonly found at each position in the compared, aligned, homologous sequences).

The consensus sequence represents a sort of <<average>> sequence which is as close as possible from all the compared sequences. For high homologous sequences or if the consensus sequence is long enough and the reaction conditions are not too stringent, it can bind to all the homologous sequences. This is especially useful for an amplification of homologous sequences with the same primers called, consensus primers. Experimentally, the consensus sequence calculated from the programs above can be adapted in order to obtain such property.

“Micro-arrays and arrays” mean solid supports on which single capture probes or capture probes species are immobilized in order to be able to bind to the given specific protein or target. The most common arrays are composed of single capture probes species being present in predetermined locations of a solid support being or not a substrate for their binding. The array is preferentially composed of spots of capture probes deposited at a given location on the surface or within the solid support or on the substrate covering the solid support. However, capture probes can be present on the solid support in various forms being but not limited to spots. One particular form of application of array is the presence of capture probes in wells having either one of several different capture probes per well and being part of the same support. Advantageously, arrays of capture probes are also provided on different supports as long as these different supports contain specific capture probes and may be distinguished from each other in order to be able to allow a quantification of a specific target sequence. This can be achieved by using a mixture of beads having particular features and being able to be recognized from each other in order to quantify the bound molecules.

The terms “capture probe” relate to molecules capable to specifically bind to a given polynucleotide or polypeptide. Preferably, polynucleotide binding is obtained through base pairing between two polynucleotides one being the immobilized capture probe or capture sequence and the other one being the target molecule (sequence) to be detected.

The term “single capture probe species” is a composition of related (poly)nucleotides for the detection of a given nucleotide sequence by base pairing hybridization. Polynucleotides are synthesized either chemically or enzymatically or purified from samples. However, the synthesis or purification is not always perfect and the capture probe can be slightly contaminated by other related molecules like shorter polynucleotides. The essential characteristic of one capture species for the invention is that the overall species can be used for capture of a given target molecule, preferably a given target nucleotide sequence.

The term “signal resulting from a specific binding at a specific location” means a detection and possibly a quantification of a single hybridization event between complementary nucleotide sequences at the specific localized area (location) of a fixed capture sequence of the solid support surface (or inside the solid support). A complementary hybridization can be detected and possibly quantified by a (fluorescent, colorimetric, etc.) label introduced in the sequence of the target sequence or at the extremity of the target sequence (preferably during the copy or amplification step) or by any of a number of means well known to those skilled in the art, such as detailed in WO 99/32660, which is incorporated herein by way of reference.

### BRIEF DESCRIPTIONS OF THE DRAWINGS

FIG. 1 is a schematic representation of a preferred method of the invention for the detection of a SNP (mutated base 1 represented by X) at given locus of gene nucleotide sequence (3) of an organism.

### DETAILED DESCRIPTION OF THE INVENTION

The present invention provides unambiguous determination of the presence or the absence of a particular base (mutated base) in a locus of a genetic sequence by examining the signal obtained in a particular location of an array which serves to question (to detect) for the presence or not of the (mutated) base in a particular locus (SNPs).

The method allows a simultaneous detection of at least 3 and better 5 and still better 20 loci and accordingly, the solid support may contain at least 6 and better 10 and still better at least 40 different capture probes having a specific sequence complementary to the different target loci to be questioned and differing by only one base (SNPs detected). The specific hybridization sequences present on the capture probes are complementary to the different targets that they correspond and they have similar chemical and physical properties. Preferably these specific sequences for on locus are identical for the target of a given locus except for the base to be questioned at the locus.

The gene nucleotide sequence(s) can be firstly extracted from the organism. Extraction means any kind of isolation of genetic material (mRNA, genomic DNA) from a sample by a physical or a chemical process. Assays on (micro)organisms extracted from a biological sample or from clinical sample or from cell culture, preferably requires an isolation of genomic DNA sequences. The different loci are either present on the same exon of a gene or on the same gene or on different genes which belong to the genome of the organism. The target sequences differing at one or at several given loci are homologous sequences.

The genetic amplification step used in the method according to the invention is performed by amplification protocols well known in the art, preferably by a method selected from the group consisting of PCR, RT-PCR, LCR, CPT, NASBA, ICR or Avalanche DNA techniques.

The nucleotide sequence of a gene is amplified using at least one primer pair (i.e. a pair of two different primers). However, several primer pairs are either used for amplifying the different specific nucleotide sequences of a gene, these sequences being preferably different exons, or used for amplifying different genes or different parts of a cell genome.

Preferably, each amplified target sequence comprises several loci. All these loci of the target are then amplified with the same primer pair being consensus primers for an amplification of all these loci, but each locus is detected on specific capture probes.

Therefore, the array contains capture probes specific for one or more loci for hybridization with target nucleotide sequence(s) comprising the mutated bases (1) to be detected in each locus, the different mutated bases being located in the same exon or in different exons originating from the same gene or from different genes, preferably present in the same nucleotide sequence (3). The amplification step of these several exons is preferably obtained with different primer pairs, each primer pair being specific for one exon. Amplification of several exons is preferably performed in the same conditions for all exons (i.e.; in the same reaction vessel or in different vessels) and are then pooled together for hybridization on a microarray. They constitute the first set of target nucleotide sequences (7 or 7′).

The second set of target nucleotide sequences (8) is amplified preferably with the same primers pairs used for the first set of target sequences (7 or 7′). In this preferred embodiment, each target sequence amplified by one primer pair (5, 6′), also contains a sequence hybridized on the second set of capture probe (10). However, the inventors have found that the requirement for the presence of the second set of target sequences on the same amplified sequence is not needed as long as that the amplifications of both target sequences using different primer pairs (5, 5′and 6, 6′) are similar. The similarity of the results is obtained by comparing the efficiency of hybridization of both amplified sequences (7, 7′,8) on the corresponding capture probes (9, 9′,10) having complementary sequences. The signals have to be identical or differing by a factor lower than 3 and preferably lower than 2.

In the next step of the method of the invention, amplified target nucleotide sequences (7 or 7′) of the first step (amplicons) are contacted with an array under conditions allowing hybridization of the target amplified sequences (7, 7′) to their complementary specific capture nucleotide sequences (9, 9′)(capture probes) present on the array. The amplicons obtained in the first step are hybridized on the same or on different arrays. In a preferred embodiment, these amplified sequences are processed (e.g. by fragmentation) prior to hybridization on the array. Fragmentation of double stranded DNA is obtained using enzymatic cleavage (DNase treatment) or chemical cleavage, preferably (depurination using HCL followed by heat denaturation as described in the US 2005/0,095,635). Fragmentation of ribonucleotide sequences is obtained by heating in the presence of magnesium ions or in the presence of KOH as described respectively in WO97/10365 and WO98/28444.

Parts (or portions) of the gene or genome sequence (loci) having possible mutations to be detected can be firstly amplified by PCR and the resulting amplicons are fragmented by DNAse treatment (Grimm et al. 2004, J.Clin. Microbiol. 42:3766-3774). In the preferred embodiment, the resulting amplicon fragments are between 30 and 70 bases long. The distribution of the fragments size obtained after fragmentation of the amplicons is advantageously checked by analysis by capillary electrophoresis (Bioanalyser, Agilent) and the average size distribution of the pieces is preferably comprised between 30 and 70 bases long.

The specific part (or portion) of the capture probes (nucleotide sequences) complementary to the target nucleotide sequence is comprised between about 10 and about 50 bases, preferably between about 15 and about 40 bases and more preferably between 18 and 24 bases. These bases are preferably assigned as a continuous sequence located at or near the extremity of the capture probes (nucleotide sequences). This sequence is considered as a specific sequence for the detection of the target nucleotide sequence.

Preferably, the melting temperature (Tm) of the specific nucleotide sequences of the capture probes (able to bind to their corresponding target nucleotide sequences) is comprised between 55 and 75° C. and preferably between 62 and 68° C. The Tm of small sequences are easily calculated from the simplified equation of Tm (° C.)=4(G+C)+2(A+T).

Preferably, the hybridization step between capture and target sequence is carried out under stringent conditions. Temperatures of hybridization is preferably between 1 and 10° C. and even preferably 1 to 4° C. lower than the Tm of the specific sequence of the capture probes. The hybridization temperature can be adapted according to the presence of molecules in the hybridization solution affecting the hybridization temperature such as the DMSO or the formamide.

Hybridization of the target sequences is performed at a temperature giving the best signal together with the lowest cross-reaction of the target on capture probes differing by one base. The optimized temperature is related on the stringency of the solution which depends on the solution composition and mainly on the salt concentration. In a preferred embodiment, the hybridization solution is composed of phosphate buffer at ph 7.4 of a molarity comprised between 0.4M to 0.8M and the hybridization temperature differs from the Tm of the specific sequences of the capture probes (9, 9′ and 10) by no more than 5° C. and preferably by no more than 2° C. In a particular embodiment the hybridization temperature is 60° C.

On the array, capture probes are arranged at pre-determined locations at a density of at least 4, 10, 16, 20, 50, 100, 1000, 4000, 10000 or more, different capture probes/cm² insoluble solid support surface. The capture probes are advantageously covalently attached to the surface of the solid support (preferably a non porous solid support surface) by one of their extremities, preferably by their 5′ end. The sensitivity may be further increased by spotting capture probes on the solid support surface by a robot at high density according to an array. The amount of capture probes spotted on the array is preferably comprised between about 0.01 to about 5 picomoles of sequence equivalent/cm² of solid support surface.

The invention is also related to a system (or kit) for the detection of a nucleotide base at a given locus of a gene nucleotide sequence of an organism (for characterizing the presence of a single nucleotide polymorphism) among at least 2 possible nucleotide bases and which incorporates media or devices for performing the method according to the invention. The kit or system is preferably included in an automatic apparatus (that performs most of the steps of the present invention automatically), such as a high throughput screening apparatus. The system or kit is adapted for performing all the steps or only several specific steps of the method according to the invention

This system (or kit) comprises:
an insoluble solid support (22) surface, preferably a non porous solid support surface comprising:
   (a) at least two capture probes (9,9′)of a first set of capture probes bound at a particular location of the insoluble solid support (22) surface, the capture probes (9,9′) having a specific sequence complementary to a portion (2) of a target nucleotide sequence (7 or 7′) comprising a first locus and wherein each capture probe (9) differs from the other capture probe (9′) by a single base (20) at the locus site, and
   (b) at least one further capture probes (10) of a second set of capture probes, being located external to the first locus and having similar physical and chemical properties as the other two capture probes (9, 9′) and having a complementary sequence to another portion (4) of the target nucleotide sequence (8), except for one base (21) not being complementary (the target and capture nucleotide sequences are complementary sequences but this complementarity differs by one base) and,
a carrier mean embedded computerized code (computer program or software) for determining (characterizing/or detecting) the nucleotide base (1) presence, at a given locus when a signal value of detection of the target sequence (7 or 7′) on their specific capture probes (9 or 9′) of the first set of capture probes is higher or equal to a cut off signal value calculated from the signal value of detection of the target sequence (8) on their specific mutated capture probe (10) of the second set of capture sequences, provides (gives) a positive result identifying (detecting the presence of) the nucleotide base (1).

In a further step, the computerized carrier mean, or computer program of the system or kit determines that the organism is heterozygote at given locus when the signal values of detection of the target nucleotide sequences (7 and 7′) upon mutated and non mutated capture probes (9 and 9′) are both positive and said signal values possibly comply with the condition that the index calculated (CI) according to the formula:

$$CI = \frac{( N 9 - N {9}^{′} )}{( N 9 + N {9}^{′} ) / 2}$$

is lower than 1, preferably lower than 0.7 and even preferably lower than 0.5;

with N9 being the average signal value of detection upon mutated (9) capture probes and N9′ being the average signal value of detection upon non mutated (9′) capture probes.

The solid support surface contains at least 10 different capture probes presented as an array targeting 5 different loci of gene(s) and better at least 40 different capture probes targeting at least 20 different loci.

In a preferred embodiment, the capture probes comprises a specific sequence for the binding to the target nucleotide sequence linked to the solid support (22) surface by a spacer (or linker) being a molecule having a physical length of at least about 6.8 nm, equivalent to the distance of at least about 20 base pair long nucleotides in double helix form or a polyethylene glycol molecule.

In a preferred embodiment, the spacer (or linker) is a polynucleotide being at least about 20 nucleotides long at least about 50 or about 70 nucleotides and preferably at least about 90 nucleotides long. The spacer (or linker) is a given nucleotide sequence being homologous to none of the genome sequence (when using an identity of at least 10 and better 5 consecutive bases). To avoid non specific hybridization, there will be no more than around 15 consecutive complementary base pair bindings between a target polynucleotide (or nucleotide) sequence and the spacer, preferably there will be less than 10 such pairings possible, more preferably less than 5. As such, the nucleotide sequence of the spacer will contain, preferably less than 15 bases and more preferably, less than 10 and still more preferably less than 5 contiguous bases complementary to the target nucleotide sequences to be detected. The determination of possible consecutive sequences is easily done by comparison of the sequences to molecular database as provided by Genbank and using software such as nucleotide-nucleotide BLAST(blastn), which can be accessed on the internet by entering the following quoted text, “www.ncbi.nlm.”, in the address bar of a web browser, such as Internet Explorer or Netscape, followed immediately by “nih.gov/BLAST”.

The total length of the capture probes (nucleotide sequences) including the spacer is comprised between about 30 and about 300 or 500 bases, preferably between about 35 and about 200 bases, more preferably between about 39 and about 120 bases.

In another preferred embodiment of the invention, capture probes (nucleotide sequences) are chemically synthesized oligonucleotide sequences of about 100 bases, which may e.g. be easily performed on programmed automatic synthesizer. Such sequences can bear a functionalized group for covalent attachment upon the support, at high concentrations. Longer capture nucleotide sequences are preferably synthesized by PCR amplification of a sequence incorporated into a plasmid containing the specific part (or portion) of the capture nucleotide sequence and the non specific part (or portion) (spacer).

Chemical and physical properties means features of the external capture probes (10) which give an efficiency of hybridization similar to the specific capture probe (9 or 9′) for the mutations to be detected.

The chemical and physical properties comprise: the specific part of the nucleotide sequence (length, Tm), the percentage of GC content and the length of the spacer.

In a preferred embodiment, the capture probe (10) external to the locus has the same features as the specific capture probes (9, 9′).

In a preferred embodiment, similar physical or chemical properties of nucleotide sequences means that the specific nucleotide sequences of the capture probes (able to bind to the target nucleotide sequences) have a Tm comprised between about 55 and about 75° C. and preferably between about 62 and about 68° C.

In a further preferred embodiment, capture probes (9, 9′) differing between them by one base have, in their specific nucleotide sequences (able to bind specifically the target nucleotide sequences) have a GC content comprised between about 40 and about 70% and preferably between about 45 and about 60%.

Furthermore, the inventors have found unexpectedly that a better discrimination of the SNP present in a locus of a gene is obtained when the nucleotide base to be detected is located at a distance of about 4 to about 10 bases and better 4 to 6 bases from one extremity of the target specific part of the bound capture probe.

The space molecule (linker) is preferably located at the 5′ extremity of the capture probe being fixed to the surface of the solid support by a covalent link present at the 5′ end or nearby. The specific nucleotide sequence for the binding to the target nucleotide sequence is preferably located at 3′ end of the capture probes (free extremity not bound to the support) at 1 to 23 nucleotides from the end.

The capture probes (9 or 9′ and 10) preferably differ by one base located at 4 to 10 and preferably at 4 to 6 bases from the (free) 3′ end of the target specific part (portion) of the bound capture probe.

The array may contain specific capture nucleotide sequences for each base of a specific locus to be detected. The bases to be detected are present within one or several exons of the same gene nucleotide sequence or from different gene nucleotide sequences.

In a preferred example, the array contains specific capture probes (9, 9′, 10) for the detection of SNP in human Cytochromes P450 2C9, 2C19 and 2D6. Cytochromes p450 2C9 and 2D6 are preferably detected upon an array of capture probes (9, 9′, 10) containing specific, as provided in table 1. Cytochrome P450 for mutations 2C9* 1, 2 and 1, 8 and for 2C 19*1, 2 and 1, 3 are also preferably added on the array together with the mutations provided in table 1.

The array may contain specific capture probes for the detection of several SNP in one gene nucleotide sequence, or the array may contain specific capture probes for the detection of several SNP in different gene nucleotide sequences.

The target nucleotide sequences are labelled during the amplification step. The labelled associated detections are numerous. A review of the different labelling molecules is given in W0 97/27317. They are obtained using either already labelled primer or by incorporation of labelled nucleotides during the amplification step. The most frequently used and preferred labels are fluorochromes like Cy3, Cy5 and Cy7 suitable for analyzing an array by using commercially available array scanners (General Scanning, Genetic Microsystem, . . . ).

Radioactive labelling, cold labelling or indirect labelling with small molecules recognized thereafter by specific ligands (streptavidin or antibodies) are common methods. The resulting signal of target fixation on the array is either fluorescent, colorimetric, diffusion, electroluminescent, bio- or chemiluminescent, magnetic, electric like impedometric or voltametric (U.S. Pat. No. 5,312,527).

A preferred method is based upon the use of the gold labelling of the bound target in order to obtain resonance light scattering (RLS) detection or silver staining which is then easily detected and quantified by a scanner. Gold particles of 10-30 nm are required for silver amplification while particles of 40-80 nm are required for direct detection of gold particles by RLS or by Photothermal Heterodyne Imaging.

In a preferred method, gold particles of 10-30 nm are amplified by silver enhancement preferably using the silverquant analysis platform including the Silverquant kit for detection, the Silverquant Scanner for slide scanning and Silverqaunt Analysis software for image quantification and data analysis (Eppendorf, Germany). Due to the non linear detection of the presence of silver, the data analysis requires a linearization of data before data processing. The data are then processed according to the invention. An algorithm of curve fitting is applied to a positive detection curve spotted on the array. Then each spot signal is linearized in ‘concentration units’ using the fitting curve.

Quantification has to take into account not only the hybridization yield and detection scale on the array but also the extraction, the amplification (or copying) and the labelling steps.

The solid support according to the invention is made with materials selected from the group consisting of gel layers, glasses, electronic devices, silicon or plastic support, polymers, compact discs, metallic supports or a mixture thereof (see EP 0 535 242, U.S. Pat. No. 5,736,257, WO99/35499, U.S. Pat No. 5,552,270, etc). Advantageously, the solid support is a single glass slide which may comprise additional means (barcodes, markers, etc.) or media for improving the method according to the invention.

In the last step of the method of the invention, results are considered positive or negative according to the fact that the signal intensity is higher or lower than a determined cut off value.

The cut off signal values are preferably taken as detected signal values of the external capture probe (10) targeting a corresponding target sequence (8) and multiplied by a factor.

In a preferred embodiment, the cut off signal value is the signal value of detection of the second set of target nucleotide sequences (8) upon its corresponding specific mutated capture probe (10) multiplied by a factor between 2 and 5 and preferably between 3 and 4, more preferably the factor 4.

In another preferred embodiment, the cut off signal value is 4 times the signal obtained with the capture probes (10) external to the detected locus, being preferably the mean value of replicated spots.

Preferably, the cut off signal value is calculated from an average signal value of detection of at least two different sets of target nucleotide sequences (8) upon at least two different complementary specific mutated capture probes (10).

The signal values of the capture probes (7 or 7′) targeting specific target loci are considered for a calculation of the cut off signal value, as long as they have the features of external capture probes (10) including differing from the target by one base.

Preferably, the cut off signal value is calculated from an average signal value of detection of at least two different sets of target nucleotide sequences (7 or 7′) upon at least two different complementary specific mutated capture probes (9 or 9′).

The method of the invention is also suited for detection of SNP in organism being heterozygote at a given locus. In a preferred embodiment, the organism is considered as heterozygote at given loci when the signal values of detection of the target nucleotide sequences (7 and 7′) upon mutated and non mutated capture probes (9 and 9′) are both positive.

The organism can also be considered as heterozygote at given loci when the signal values of detection of the target nucleotide sequences (7 and 7′) upon mutated and non mutated capture probes (9 and 9′) are both positive and furthermore that said signal values comply with the condition that the index calculated (CI) according to the formula:

$$CI \frac{( N 9 - N {9}^{′} )}{( N 9 + N {9}^{′} ) \text{/} 2}$$

is lower than 1;
with N being the average signal value of detection upon mutated (9) or non mutated (9′) capture probes. In a still preferred embodiment the CI is lower than 0.7 and even better lower than 0.5.

In a preferred embodiment, the organism is considered as homozygote at a given locus when only one of the signal values of detection of the target nucleotide sequences (7 or 7′) upon mutated and non mutated capture probes (9 or 9′) is positive.

Advantageously, the array also contains capture probes for the specific detection of the specifically amplified target gene(s) and being located outside the locus of interest. Such capture probes are preferably added for confirmation of the identification of gene(s) or for discrimination between two genes being very homologous. The capture probe is used as positive control of the PCR and hybridization.

Advantageously, the array also contains spots with various concentrations (i.e. 4) of labelled capture probes. These labelled capture probes are spotted from known concentrations solutions and their signals allow the conversion of the results of hybridization into absolute amounts. They also allow testing for the reproducibility of the detection.

In one embodiment, the solid support (biochip) is inserted in a support connected in and out pipes and handled by an automatic machine controlling liquid solution as being developed in the microfluidic technology. By being inserted into such a microlaboratory system, it can be incubated, heated, washed and labelled by automates, even for previous steps (like extraction of DNA, amplification by PCR) or the following step (labelling and detection). All these steps can be performed upon the same solid support.

Preferably, the kit of the invention comprises at least an insoluble solid support upon which are bound some capture probes (preferably bound to the surface of the solid support by a direct covalent link or by the intermediate of a spacer) according to an array with a density of at least 4, preferably at least 10, 16, 20, 50, 100, 1000, 4000, 10 000 or more, different capture probes(s)/cm² insoluble solid support surface, said capture probes having advantageously a length comprised between about 30 and about 300 bases (including the spacer) and containing a sequence of about 10 to about 50 bases, said sequence being specific for the target (which means that said bases of said sequence are able to form a binding with their complementary bases upon the sequence of the target by complementary hybridization).

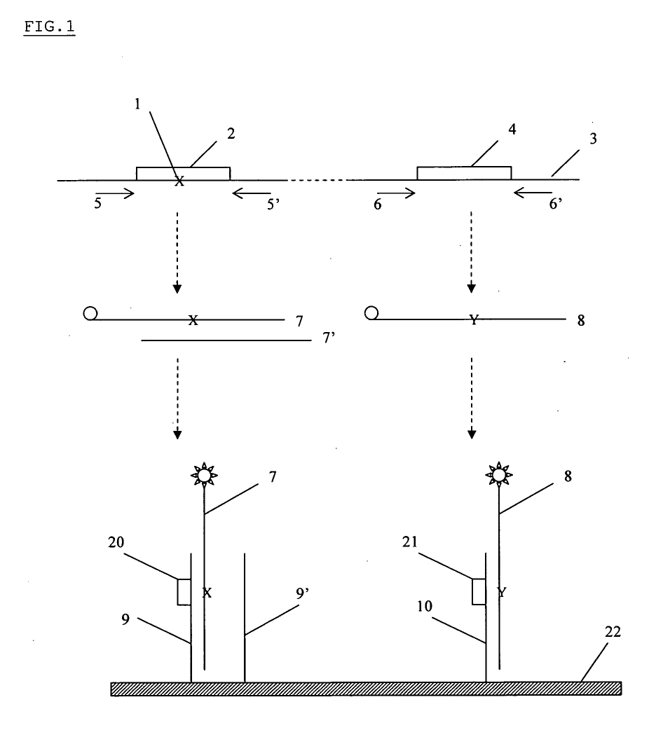

FIG. 1 is a schematic representation of a preferred method of the invention for the detection of a SNP (mutated base 1 represented by X) at given locus of gene nucleotide sequence (3) of an organism. A portion of the gene nucleotide sequence is amplified by PCR using either a single primer pair (5, 6′) or different primer pairs (5, 5′ and 6, 6′) into a first set of target nucleotide sequences (7 or 7′) and a second set of target nucleotide sequence (8) which are both labelled (circle). The amplicons, after labelling, are then contacted with an array of capture probes immobilized on a solid support (22) comprising : (a) a pair of capture probes (9 and 9′) having the same specific hybridization sequence complementary to a portion (2) of the target nucleotide sequence (7 and 7′) but differing by a single base (20) corresponding to the mutated base (1) present at the locus to be characterized; (b) a capture probe (10) having a specific hybridization sequence complementary to a portion (4) of the target nucleotide sequence (8) but differing by single base (21) at position Y. The presence of SNP is determined if the signal value of detection of the target sequence (7 and/or 7′) upon their corresponding capture probes (9 or 9′) is higher or equal to a cut off signal value of detection of the target sequence (8) upon its corresponding specific mutated capture probe (10). All capture probes have similar physical and chemical properties.

Table 1 presents the list of probes for SNP detection on CYP2D6 and CYP2C9 human genes. Sequence, melting temperature (Tm), GC percentage (% GC) and length of each probe are detailed. The substituted nucleotides are underlined; the points represent deleted nucleotides.

Table 2 presents the result of SNP detection of a sample CYP2D6*1/*4 combined with CYP2C9*1/*3. The sample have been hybridized 5 times (n=5).

Table 3 presents the result of SNP detection of a sample CYP2C9*9. The sample have been hybridized 3 times (n=3).

### EXAMPLES

### Example 1

### Detection of 2 mutations in CYP2C9 (exon 3)

The CYP2C9 gene containing 2 main mutations in the exon 3 is amplified by PCR using the following primers:

(SEQ ID NO: 1)

| | |
|---|---|
| MP2C19 06: | 5′ AGCAATGGAAAGARATGGAAGGAG 3′ |

(SEQ ID NO: 2)

| | |
|---|---|
| MP2C9 07: | 5′ GAAACCCCAGAGAAGTCAGTGAG 3′ |

The PCR was performed on genomic DNA extracted from blood sample with the kit Quiaamp DNA Blood mini (Qiagen, Venlo, Netherlands) in a final volume of 50 μl containing: 3 mM MgCl_{2,} 10 mM Tris pH 8.4, 50 mM KCl, 125 μM of each primer, 200 μM of dATP, dCTP and dGTP (Roche), 150 μM of dTTP, 50 μM of dUTP, 10 μM of biotin-11-dATP (PerkinElmer), 10 μM of biotin-11-dCTP (PerkinElmer), 2.5 U of Taq DNA polymerase Ultratools (Labsystems), 25 ng of genomic DNA. Samples were first denatured at 94° C. for 5 min. Then 40 cycles of amplification were performed consisting of 30 sec at 94° C., 1 min at 63° C. and 1 min at 72° C. and a final extension step of 10 min at 72° C. Water controls were used as negative controls of the amplification. The expected size is 622 bp.

Capture Nucleotide Sequence Immobilization

The protocol described in WO02/18288 (example 1 and 2) was followed for obtaining aldehyde derivatized glass slide (diaglass slide). A list of capture probes for SNP detection on CYP2D6 and CYP2C9 human genes are presented in table 1. The aminated capture nucleotide sequences were spotted on diaglass slide according to the protocol described in WO01/77372 (example 1) at a concentration of 3000 nM.

Fragmentation

After the PCR, the amplified sequences (amplicons) are purified, quantified. The amplified DNA are fragmented with a DNAse (Promega, Madison, USA) at room temperature for 5 min. The reaction are stopped by the addition of 3 mM EGTA and incubation at 65° C. for 10 min (Grimm et al. 2004, J Clin. Microbiol., 42, 3766-3774). The DNAse cuts the amplicons randomly giving rise to fragments of approximately 50 bp. The efficiency of the fragmentation is checked using a DNA 1000 LabChip® (Agilent technologies, Germany).

Hybridization

A volume of 10 μl of NaOH 0.175 N was added at 10 μl of fragmentated amplicons, 5 μl of positive hybridization control and 10 μl of distilled water. The mix was incubated at room temperature for 5 min. A volume of 35 μl of Genomic Hybribuffer (Eppendorf, Germany) was finally added and the solution was loaded on the array framed by a hybridization chamber. The chamber was closed with a coverslip. The hybridization was carried out at 60° for 2 h. Samples were washed 4 times with Unibuffer (Eppendorf, Germany).

Colorimetric Detection

The glass samples were incubated 45 min at room temperature with colloidal gold-conjugated IgG Anti-biotin 1000×diluted in blocking buffer. After 5 washes with washing buffer, the presence of gold served for catalysis of silver reduction using the Silverquant kit (Eppendorf, Hamburg, Germany). The slides were incubated 1 times 5 min with the revelation mixture of Silverquant A and B solutions, then rinsed with water, dried and analyzed using the Silverquant scanner (Eppendorf, Hamburg, Germany). Each slide was then quantified by the silverquant Analysis software. A curve fitting algorithm was used to linearize the signal intensities obtained with silverquant detection method. The algorithm used a positive detection curve spotted on the array.

Fluorescence Detection

The glass samples were incubated 45 min at room temperature with the Cy3-conjugated IgG Anti-biotin (Jackson Immuno Research Laboratories, Inc #200-162-096) diluted 1/1000 Conjugate-Cy3 in the blocking buffer and protected from light. After washing the slides were dried before being stored at room temperature. The detection was performed in the laser confocal scanner “ScanArray”™ (Packard, USA) Each slide was then quantified by the Imagene quantification software (Biodiscovery).

Data Analysis

The signals are compared to a threshold capture probe which is the mutated external Hybridization probe. If the signal is higher than the threshold probe multiplied by a factor 4, the result is considered as positive. Inversely, if the signal is lower, the result is considered as negative.

### Example 2

### Detection of Multiple Mutations in CYP2D6 and CYP2C9 (exons 5 and 7)

The CYP2D6 gene containing 3 main mutations and the CYP2C9 gene containing 6 main mutations in the exons 5 and 7 were amplified by PCR using the following primers:

(SEQ ID NO: 3)

MP2D6 01: 5′ CGCTTCTCCGTGTCCACCTTGC 3′

(SEQ ID NO: 4)

MP2D6 02: 5′ GCCAGTCTCACCTTCTCCATCTC 3′

(SEQ ID NO: 5)

MP2C9 06: 5′ GCTTTGTACTATCAATCAGGTTGTC 3′

(SEQ ID NO: 6)

MP2C9 02: 5′ CACAAATTCACAAGCAGTCACATAAC 3′

(SEQ ID NO: 7)

MP2C9 03: 5′ CTAAAGTCCAGGAAGAGATTGAACG 3′

(SEQ ID NO: 8)

MP2C9 04: 5′ CAGAGTGTTGATTTGACAAGATTTTAC 3′

The expected sizes of the amplicons were 1014 bp for CYP2D6, 626 bp for CYP2C9 exon 5 and 1114 bp for CYP2C9 exon 7.

PCRs, fragmentations, hybridizations, detection were performed as described in the example 1 using fluorescence detection. The specific parts of the capture probes for hybridization of the targets are presented in table 1. This table comprises capture probes for the detection of 9 mutations as well as external capture probes (10) of the invention (2D6 and 2C9 external probes) corresponding to each amplified exon. The array also contains control probes which are the non mutated external probes of the invention (2D6 and 2C9 control probes). The analysis was performed on several clinical samples and the data presented in table 2 and 3. The results were analyzed as proposed in the invention and the final results were compared with the determination of the sample mutations by sequencing. There was 100% correlation of the results.

TABLE 1

List of probes for SNP detection on CYP2D6 and
CYP2C9 genes (SEQ ID NOs: 9-30). Sequence, melting
temperature (Tm), GC percentage (% GC) and length
of each probe are detailed. The substituted nucleotides
are underlined; the points represent deleted nucleotides.

| | | | | |
|---|---|---|---|---|
| | | Tm | | |
| Mutation | Sequence | (° C.) | % GC | Length |

| | | | | | |
|---|---|---|---|---|---|
| 2D6*1, 3 | CTGCTAACTGAGCACAGGATG | (SEQ ID NO: 9) | 64 | 52, 4 | 21 |
| 2D6*3 | CTGCTAACTGAGCAC.GGATGA | (SEQ ID NO: 10) | 64 | 52, 4 | 21 |
| 2D6*1, 4 | GCGAAAGGGGCGTCCTGGG | (SEQ ID NO: 11) | 66 | 73, 7 | 19 |
| 2D6*4 | GCGAAAGGGGCGTCTTGGG | (SEQ ID NO: 12) | 64 | 68, 4 | 19 |
| 2D6*1, 6 | CCTCGGTCACCCACTGCTC | (SEQ ID NO: 13) | 64 | 68, 4 | 19 |
| 2D6*6 | TCCTCGGTCACCC.CTGCTC | (SEQ ID NO: 14) | 64 | 68, 4 | 19 |
| 2D6 control | GTTGGCGAAGGCGGCACAAA | (SEQ ID NO: 15) | 64 | 60 | 20 |
| probe | | | | | |
| 2D6 external | GTTGGCGAAGGCGGAACAAAG | (SEQ ID NO: 16) | 66 | 57, 1 | 21 |
| probe | | | | | |
| 2C9*1, 9 | TGTCCATTGATTCTTGGTGTTC | (SEQ ID NO: 17) | 62 | 40, 9 | 22 |
| 2C9*9 | GTCCATTGATTCTTGGCGTTC | (SEQ ID NO: 18) | 62 | 47, 6 | 21 |
| 2C9*1, 10 | CTTCCTGATGAAAATGGAGAAGG | (SEQ ID NO: 19) | 66 | 43, 5 | 23 |
| 2C9*10 | CTTCCTGATGAAAATGGGGAAGG | (SEQ ID NO: 20) | 68 | 47, 8 | 23 |
| 2C9*1, 3 = *1, 4 | GGTGGGGAGAAGGTCAATGTA | (SEQ ID NO: 21) | 64 | 52, 4 | 21 |
| 2C9*3 | GGTGGGGAGAAGGTCAAGGTA | (SEQ ID NO: 22) | 66 | 57, 1 | 21 |
| 2C9*1, 4 | GGTGGGGAGAAGGTCAATGTA | (SEQ ID NO: 23) | 64 | 52, 4 | 21 |
| 2C9*4 | GGTGGGGAGAAGGTCAGTGTA | (SEQ ID NO: 24) | 66 | 57, 1 | 21 |
| 2C9*1, 5 | GCTGGTGGGGAGAAGGTCAA | (SEQ ID NO: 25) | 64 | 60 | 20 |
| 2C9*5 | GCTGGTGGGGAGAAGCTCAA | (SEQ ID NO: 26) | 64 | 60 | 20 |
| 2C9*1, 11 | ATGCAGGGGCTCCGGTTTCT | (SEQ ID NO: 27) | 64 | 60 | 20 |
| 2C9*11 | CATGCAGGGGCTCCAGTTTC | (SEQ ID NO: 28) | 64 | 60 | 20 |
| 2C9 control | GCAGGCTGGTGGGGAGAAG | (SEQ ID NO: 29) | 64 | 68, 4 | 19 |
| probe | | | | | |
| 2C9 external | GCAGGCTGGTGGGAAGAAG | (SEQ ID NO: 30) | 62 | 63, 2 | 19 |
| probe | | | | | |

TABLE 2
Sample CYP2D6*1/*4 combined with CYP2C9*1/*3. The
sample have been hybridized 5 times (n = 5).

| | Mean values | Standard | Results | Results |
|---|---|---|---|---|
| Mutation | n = 5 | Deviation | Invention | Sequencing |
| 2D6*1,3 | 14769 | 2799 | + | + |
| 2D6*3 | 130 | 130 | − | − |
| 2D6*1,4 | 14025 | 2926 | + | + |
| 2D6*4 | 15354 | 2562 | + | + |
| 2D6*1,6 | 51862 | 4289 | + | + |
| 2D6*6 | 1164 | 174 | − | − |
| 2D6 control probe | 20273 | 3595 | + | + |
| 2D6 external probe | 729 | 221 | − | − |
| 2C9*1,9 | 16664 | 2731 | + | + |
| 2C9*9 | 681 | 111 | − | − |
| 2C9*1,10 | 20239 | 2929 | + | + |
| 2C9*10 | 1090 | 140 | − | − |
| 2C9*1,3 = *1,4 | 33802 | 4299 | + | + |
| 2C9*3 | 28589 | 2903 | + | + |
| 2C9*1,4 | 33803 | 4299 | + | + |
| 2C9*4 | 2342 | 247 | − | − |
| 2C9*1,5 | 53000 | 9404 | + | + |
| 2C9*5 | 368 | 111 | − | − |
| 2C9*1,11 | 65061 | 165 | + | + |
| 2C9*11 | 1990 | 224 | − | − |
| 2C9 control probe | 65156 | 83 | + | + |
| 2C9 external probe | 818 | 183 | − | − |

TABLE 3
Sample CYP2C9*9. The sample have been
hybridized 3 times (n = 3).

| | Mean values | Standard | Results | Results |
|---|---|---|---|---|
| Mutation | n = 3 | Deviation | Invention | Sequencing |
| 2D6*1,3 | 16055 | 784 | + | + |
| 2D6*3 | 795 | 57 | − | − |
| 2D6*1,4 | 29935 | 334 | + | + |
| 2D6*4 | 2566 | 196 | − | − |
| 2D6*1,6 | 44038 | 2992 | + | + |
| 2D6*6 | 1012 | 115 | − | − |
| 2D6 control probe | 33486 | 1150 | + | + |
| 2D6 external | 1285 | 51 | − | − |
| probe | | | | |
| 2C9*1,9 | 437 | 15 | − | − |
| 2C9*9 | 13084 | 556 | + | + |
| 2C9*1,10 | 15250 | 1015 | + | + |
| 2C9*10 | 731 | 87 | − | − |
| 2C9*1,3 = *1,4 | 41655 | 2258 | + | + |
| 2C9*3 | 1953 | 73 | − | − |
| 2C9*1,4 | 41655 | 2258 | + | + |
| 2C9*4 | 2449 | 171 | − | − |
| 2C9*1,5 | 53196 | 4270 | + | + |
| 2C9*5 | 253 | 14 | − | − |
| 2C9*1,11 | 65048 | 65 | + | + |
| 2C9*11 | 1619 | 142 | − | − |
| 2C9 control probe | 65172 | 13 | + | + |
| 2C9 external | 617 | 80 | − | − |
| probe | | | | |

What is claimed is:

1. A method to determine a presence or absence of at least 3 single nucleotide polymorphisms or SNP(s) (mutated base) at given loci of gene nucleotide sequence(s) of an organism and comprising the steps of:
amplifying the organism obtained from homologous gene nucleotide sequence(s) or a portion, thereof containing different loci into a first set(s) of target nucleotide sequences and;
amplifying a second fragment of gene nucleotide sequence(s) being non mutated into a second set of target nucleotide sequences;
putting into contact the first set(s) of target nucleotide sequences upon an array of capture probes wherein the different capture probes have similar physical and chemical properties, and wherein the said array comprises a series of pair of capture probes, having the same specific hybridization sequence complementary to a portion of the different target nucleotide sequences , but differing by a single base, the single base being complementary to the mutated base present in the locus sequence to be characterized;
putting into contact the second set of target nucleotide sequences with a complementary capture probe having a specific hybridization sequence complementary to a portion of the said target nucleotide sequence but differing by a single base;
detecting and/or quantifying a signal value of hybridization of the first set of target nucleotide sequences and a signal value of hybridization of the second set of target nucleotide sequences;
determining the presence of the nucleotide base at the different said loci when the signal values of detection of the first set of the target sequence(s) upon their corresponding capture probes are higher or equal to a cut off signal value calculated from the signal value of detection of the second set of the target sequences upon corresponding specific mutated capture probe; and
determining the presence of a single nucleotide polymorphisms (of a mutated base) in the different said loci.

2. The method according to claim 1, wherein similar physical and chemical properties of the capture probes have a GC content comprised between about 40 and about 70%.

3. The method according to claim 1, wherein the length of the capture probe is comprised between about 15 and about 40 bases.

4. The method according to claim 1, wherein the capture probes have a Tm comprised between about 55 and about 75° C.

5. The method according to claim 1, wherein the hybridization temperature is 1 and 10° C., and is lower than the melting temperature (Tm) of the specific sequence of the capture probes.

6. The method according to claim 1, wherein the capture probes are covalently attached to the surface of the solid support surface by their 5′ end.

7. The method according to claim 1, wherein the capture probes differ by one base located at a distance of about 4 to about 10 from the 3′ end of the target specific part of the bound capture probe.

8. The method according to claim 1, wherein the cut off signal value is the signal value of detection of the second set of target nucleotide sequences upon corresponding specific mutated capture probe multiplied by a factor between 2 and 5.

9. The method of the claim 8, wherein the signal value of detection is multiplied by the factor 4.

10. The method according to claim 8, wherein the cut off signal value is calculated from an average signal value of detection of at least two different sets of target nucleotide sequences of the second set of target nucleotide sequences upon at least two different complementary specific mutated capture probes.

11. The method according to claim 1, wherein the organism is considered as heterozygote at given loci, when the signal values of detection of the target nucleotide sequences of the first set of target nucleotide sequences upon mutated and non mutated capture probes are both positive.

12. The method according to claim 11, wherein the organism is considered as heterozygote at given loci when the signal values of detection of the target nucleotide sequences of the first set of target nucleotide sequences upon mutated and non mutated capture probes are both positive and furthermore when said signal values comply with the condition that the index calculated according to the formula:
$$CI \frac{( N 9 - N {9}^{′} )}{( N 9 + N {9}^{′} ) \text{/} 2}$$
is lower than 1,
wherein N9 is the average signal value of detection upon mutated capture probes and capture probes N9′ is the average signal value of detection upon non mutated capture probes.

13. The method according to claim 12, wherein the CI is lower than 0.7.

14. The method according to claim 1, wherein the first and second sets of target nucleotide sequences are obtained by amplification with the same primer pair.

15. The method according to claim 1 , wherein the first and second sets of target nucleotide sequences obtained by amplification with different primer pairs provide a detection signal on specific capture probes which are identical or differing by a factor lower than 3.

16. The method according to claim 1, for a detection of SNP in human Cytochromes P450 2C9, 2C19 and 2D6.

17. The method according to claim 16, wherein the cytochromes p450, 2C9 and 2D6 are detected upon an array of capture probes containing specific sequences as provided in table 1.

18. The method according to claim 1, wherein the target nucleotide sequences are labelled during amplification.

19. The method according to claim 1, wherein the target nucleotide sequences are fragmented after amplification.

20. The method according to claim 1, wherein the target nucleotide sequences are fragmented by DNase treatment after amplification.

21. The method according to claim 1, wherein the capture probes comprises a specific sequence for the binding to the target nucleotide sequence linked to the solid support surface by a spacer.

22. The method according to claim 21, wherein the spacer is a polynucleotide of at least about 20 nucleotides long.

23. The method according to claim 21, wherein the spacer is a polynucleotide of at least about 90 nucleotides long.

24. The method of claim 1, further comprising an extracting step of the gene nucleotide sequence(s) from the organism.

25. A system for detection of a nucleotide base at a given locus of a nucleotide sequence of an organism comprising:
an insoluble solid support surface, comprising:
at least two capture probes of a first set of capture probes bound at particular locations of the insoluble solid support surface, said capture probes having a specific sequence complementary to a portion of a target nucleotide sequence comprising a first locus and wherein each capture probe differs from the other capture probes by a single base at the locus site;
at least one further capture probe of a second set of capture probes being located external to the first locus and having similar physical and chemical properties as the other two capture probes and having a complementary sequence to another portion of the target nucleotide sequence except for one base not being complementary; and
a computer or a carrier mean embedded computerized code for determining or detecting the nucleotide base presence at a given locus by a signal value of detection of the target sequence on specific capture probes of the first set of capture probes, which is higher or equal to a cut off signal value calculated from a signal value of detection of the target sequence on specific mutated capture probes of the second set of capture probes that gives a positive result identifying the nucleotide base.

26. The system according to claim 25, wherein the insoluble solid support surface contains at least 10 different capture probes targeting 5 different loci of gene(s).

27. The system according to any of the claim 25, wherein the capture probes differ by one base located at 4 to 10 from one extremity of the target specific part of the bound capture probe.

28. The system according to any of the claim 25, wherein the capture probes are covalently attached to the surface of the solid support by their 5′ end.

29. The system according to any of the claim 25, wherein the computer program or carrier mean embedded computerized code determines that the organism is heterozygote at given locus when the signal values of detection of the target nucleotide sequences upon mutated and non mutated capture probes of the first set of capture probes are both positive.

30. The system according to claim 29, wherein the computer program or carrier mean embedded computerized code determines that the organism is heterozygote at given locus when the signal values of detection of the target nucleotide sequences upon mutated capture probes and non mutated capture probes of the first set of capture probes are both positive and furthermore that said signal values comply with the condition that the index calculated according to the formula:
$$CI = \frac{( N 9 - N {9}^{′} )}{( N 9 + N {9}^{′} ) / 2}$$
is lower than 1;
wherein N9 is the average signal value of detection upon mutated capture probes and wherein N9′ is the average signal value of detection upon non-mutated capture probes.


SEQUENCE LISTING (30 sequences)

SEQ ID NO: 1 - DNA, 24 bp, Artificial Sequence
  MP2C19 06 primer
agcaatggaaagaratggaaggag

SEQ ID NO: 2 - DNA, 23 bp, Artificial Sequence
  MP2C9 07 primer
gaaaccccagagaagtcagtgag

SEQ ID NO: 3 - DNA, 22 bp, Artificial Sequence
  MP2D6 01 primer
cgcttctccgtgtccaccttgc

SEQ ID NO: 4 - DNA, 23 bp, Artificial Sequence
  MP2D6 02 primer
gccactctcaccttctccatctc

SEQ ID NO: 5 - DNA, 25 bp, Artificial Sequence
  MP2C9 06 primer
gctttgtactatcaatcaggttgtc

SEQ ID NO: 6 - DNA, 26 bp, Artificial Sequence
  MP2C9 02 primer
cacaaattcacaagcagtcacataac

SEQ ID NO: 7 - DNA, 25 bp, Artificial Sequence
  MP2C9 03 primer
ctaaagtccaggaagagattgaacg

SEQ ID NO: 8 - DNA, 27 bp, Artificial Sequence
  MP2C9 04 primer
cagagtgttgatttgacaagattttac

SEQ ID NO: 9 - DNA, 21 bp, Artificial Sequence
  2D6*1,3 probe
ctgctaactgagcacaggatg

SEQ ID NO: 10 - DNA, 21 bp, Artificial Sequence
  2D6*3 probe
ctgctaactgagcacggatga

SEQ ID NO: 11 - DNA, 19 bp, Artificial Sequence
  2D6*1,4 probe
gcgaaaggggcgtcctggg

SEQ ID NO: 12 - DNA, 19 bp, Artificial Sequence
  2D6*4 probe
gcgaaaggggcgtcttggg

SEQ ID NO: 13 - DNA, 19 bp, Artificial Sequence
  2D6*1,6 probe
cctcggtcacccactgctc

SEQ ID NO: 14 - DNA, 19 bp, Artificial Sequence
  2D6*6 probe
tcctcggtcacccctgctc

SEQ ID NO: 15 - DNA, 20 bp, Artificial Sequence
  2D6 control probe
gttggcgaaggcggcacaaa

SEQ ID NO: 16 - DNA, 21 bp, Artificial Sequence
  2D6 external probe
gttggcgaaggcggaacaaag

SEQ ID NO: 17 - DNA, 22 bp, Artificial Sequence
  2C9*1,9 probe
tgtccattgattcttggtgttc

SEQ ID NO: 18 - DNA, 21 bp, Artificial Sequence
  2C9*9 probe
gtccattgattcttggcgttc

SEQ ID NO: 19 - DNA, 23 bp, Artificial Sequence
  2C9*1,10 probe
cttcctgatgaaaatggagaagg

SEQ ID NO: 20 - DNA, 23 bp, Artificial Sequence
  2C9*10 probe
cttcctgatgaaaatggggaagg

SEQ ID NO: 21 - DNA, 21 bp, Artificial Sequence
  2C9*1,3=*1,4 probe
ggtggggagaaggtcaatgta

SEQ ID NO: 22 - DNA, 21 bp, Artificial Sequence
  2C9*3 probe
ggtggggagaaggtcaaggta

SEQ ID NO: 23 - DNA, 21 bp, Artificial Sequence
  2C9*1,4 probe
ggtggggagaaggtcaatgta

SEQ ID NO: 24 - DNA, 21 bp, Artificial Sequence
  2C9*4 probe
ggtggggagaaggtcagtgta

SEQ ID NO: 25 - DNA, 20 bp, Artificial Sequence
  2C9*1,5 probe
gctggtggggagaaggtcaa

SEQ ID NO: 26 - DNA, 20 bp, Artificial Sequence
  2C9*5 probe
gctggtggggagaagctcaa

SEQ ID NO: 27 - DNA, 20 bp, Artificial Sequence
  2C9*1,11 probe
atgcaggggctccggtttct

SEQ ID NO: 28 - DNA, 20 bp, Artificial Sequence
  2C9*11 probe
catgcaggggctccagtttc

SEQ ID NO: 29 - DNA, 19 bp, Artificial Sequence
  2C9 control probe
gcaggctggtggggagaag

SEQ ID NO: 30 - DNA, 19 bp, Artificial Sequence
  2C9 external probe
gcaggctggtgggaagaag